(19) United States
(12) Patent Application Publication
(10) Pub. No.: US 2001/0055766 A1
(43) Pub. Date: Dec. 27, 2001

(54) Immunosorbant assay using branched bis-biotin/avidin/multiple label complex as a detection reagent

(76) Inventors: Alexander Aristarkhov, Brookline, MA (US); Michelle A.J. Palmer, Arlington, MA (US)

Correspondence Address: Supervisor, Patent Prosecution Services, PIPER MARBURY RUDNICK & WOLFE LLP, 1200 Nineteenth Street, N.W., Washington, DC, 20036-2412, US

(21) Appl. No.: 09/802,902

(22) Filed: Mar. 12, 2001

Related U.S. Application Data
(63) Continuation-in-part of application No. 09/540,496, filed on Mar. 31, 2000, now abandoned.
(60) Provisional application No. 60/127,480, filed on Apr. 2, 1999.
(60) Provisional application No. 60/169,618, filed on Dec. 8, 1999.

(51) Int. Cl. C12Q 1/68; G01N 33/53

(52) U.S. Cl. 435/6; 435/7.5

(57) ABSTRACT

The present invention relates to a branched bis-biotin/avidin/multiple label complex that is conjugated to a member of a specific binding pair (“sbp member”). The complex and conjugate compositions of the invention find use in an assay for an analyte wherein there is employed a reagent system comprising an avidin reagent and a biotin reagent. The present invention comprises using as the biotin reagent the branched bis-biotin/avidin/multiple label complex as described above. Also disclosed are kits comprising the present bis-biotin/avidin/multiple label complex and methods of preparing a bis-biotin/avidin/multiple label complex conjugate of a member of a specific binding pair (“sbp member”) for use in a specific binding assay.

This application is a continuation-in-part of U.S. application Ser. No. 09/540,496 filed Mar. 31, 2000, which in turn claims priority from U.S. Provisional Application Ser. No. 60/127,480 filed Apr. 2, 1999 and No. 60/169,618 filed Dec. 8, 1999. The entire contents of each of the aforementioned applications are incorporated herein by reference.

### BACKGROUND OF THE INVENTION

1. Field of the Invention

The present invention relates generally to methods for performing biological assays, including immunoassays, receptor assays, nucleic acid detection assays and related assays. The present invention relates in particular to branched bis-biotin/avidin/multiple label complexes and compositions containing the bis-biotin/avidin/multiple label complexes that find use, for example, in assays for analytes, such as, e.g., immunoassays, receptor assays and nucleic acid detection assays. In such assays, it is often necessary to bind together two components, one being, for example, a specific binding pair member and the other being another assay component such as, for example, surfaces, antigens, haptens, nucleic acids and proteins such as antibodies, etc.

2. Related Art

It is known to use reagents containing biotin in bioassays. Such biotin reagents generally have one of two components to be bound or conjugated to biotin. An avidin reagent that has avidin or streptavidin bound to the other of the two components to be bound is generally also employed. The biotin reagent is combined with the avidin reagent via interactions between the biotin and avidin, including hydrogen bonds and van der Waals interactions.

Typically, in such assays, a target sample is inspected for the presence and/or amount of a particular analyte. Immunoassays, receptor assays and nucleic acid detection assays require that detection be specific, i.e., a positive indication should be given only because of the presence of target analyte and not because of other factors. The detection signal must be strong and easily determined.

Wilbur et al., Bioconjugate Chem. 8:819-832 (1997), disclose an investigation of polymerization and/or cross-linking of recombinant streptavidin with biotin dimers and trimers as a model for reagents to be used to increase the amount of radioactivity on cancer cells. As reported therein, none of the biotin dimers caused polymerization, while all of the biotin trimers resulted in complete polymerization. Similarly, only a very small amount, if any, of cross-linking with recombinant streptavidin was obtained using biotin dimers, while biotin trimers provided significant cross-linking.

U.S. Pat. No. 4,298,685 discloses a diagnostic reagent for use in assays. The reagent is a conjugate of biotin and antibodies for the substance to be determined in the assay.

U.S. Pat. No. 5,219,764 discloses hapten-biotin conjugates, wherein the hapten is linked to biotin by means of a spacer, which has 26 to 40 atoms in its chain and contains at least five heteroatoms. The conjugates find use in certain homogeneous immunoassays.

Green, Methods Enzymol. 184:51-67 (1990), discloses avidin and streptavidin complexes.

Green et al., Biochem. J. 125:781-791 (1971), disclose the use of bifunctional biotinyl compounds to determine the arrangement of subunits in avidin.

Weber et al., Science 243:85-88 (1989), disclose structural origins of highaffinity biotin binding to streptavidin.

Morgan et al., Polym. Sci., Part A: Polym. Chem. 32:1331-1340 (1994), relates to the synthesis of aromatic bis-biotin ligands and their ability to polymerize with avidin and streptavidin.

Immunopure® ABC Staining Kits from Pierce of Rockford, Ill. include avidin and biotinylated enzyme reagents. In use, these kits mix the components together on the surface. Any resulting polymerization is not, therefore, controllable.

Pierlot et al., Bioorg. Med. Chem. Lett. 2:267-271 (1992), disclose solid phase synthesis of 5′ non-radioactive multiple labeled oligodeoxyribonucleotides.

U.S. Pat. No. 6,153,442 discloses bis-biotin compounds, complexes of bis-biotin compounds and uses for the complexes in bioassays. The bis-biotin compounds contain two biotin radicals connected to a chain of at least 16 atoms in length and having a 1,3-disubstituted aromatic ring as part of the chain.

There exists a need in the art for a controllable and reproducible polymerization of biotin and avidin or streptavidin. A need also exists for amplification of the assay detection signal (i.e., signal amplification).

### SUMMARY OF THE INVENTION

It is an object of the present invention to provide an assay system and method for detecting a target analyte having an enhanced detectable signal.

Another object of the present invention is to provide polymer particles of certain reproducible size to use in reactions as a detection reagent for a target analyte.

In accordance with the above object, the present invention includes a method for preparing a branched bis-biotin/avidin/multiple label complex. The method further comprises preparing a bis-biotin/avidin/multiple label complex conjugate composition comprising reacting a specific binding pair member with the bis-biotin/avidin/multiple label complex. The resulting composition, which contains more than one label moiety within the complex, provides for enhanced signal amplification.

The present invention is also directed to the bis-biotin/avidin/multiple label complex formed by the method of the present invention. The branched bis-biotin/avidin/multiple label complex comprises layers of bis-biotin and free biotin attached to avidin and two or more labeled moieties attached to the outer layer of avidin via a biotin moiety. The bis-biotin/avidin/multiple label complex is conjugated to a member of a specific binding pair to form a detection reagent.

Another aspect of the present invention is a composition comprising the bis-biotin/avidin/multiple label complex conjugated to a specific binding pair member.

Yet another aspect of the present invention is an assay for an analyte wherein the bis-biotin/avidin/multiple label complex is employed as a reagent system to bind specific binding pair members.

A further aspect of the present invention is a kit comprising, in packaged combination, the bis-biotin/avidin/multiple label complex and one of the specific binding pair members.

### DESCRIPTION OF THE FIGURES

FIG. 1 shows a molecular depiction of the bis-biotin/avidin/multiple label-specific binding pair conjugate composition in accordance with the present invention.

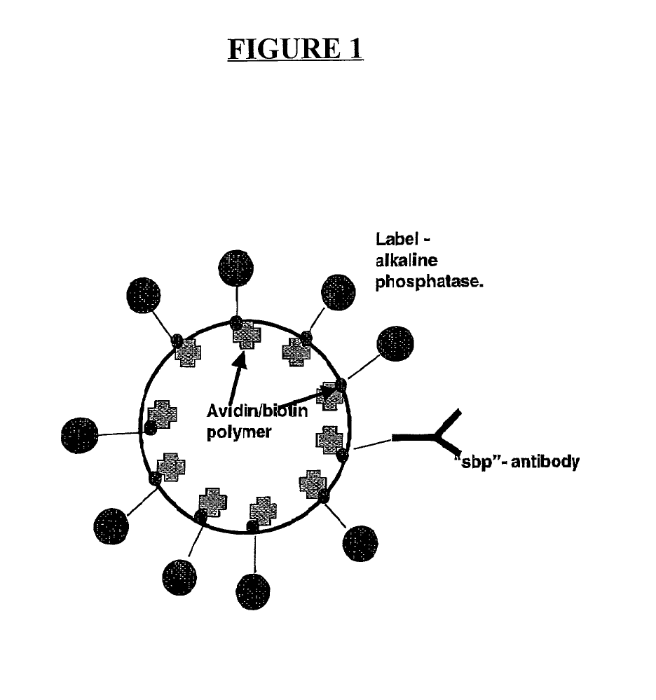

FIG. 2 shows the formation of the bis-biotin/avidin/multiple label-specific binding pair conjugate composition in accordance with the present invention.

FIG. 3 shows the steps involved in an enzyme-linked immunosorbent assay (ELISA)-type assay using the inventive bis-biotin/avidin/multiple label-specific binding pair conjugate composition as a detection reagent.

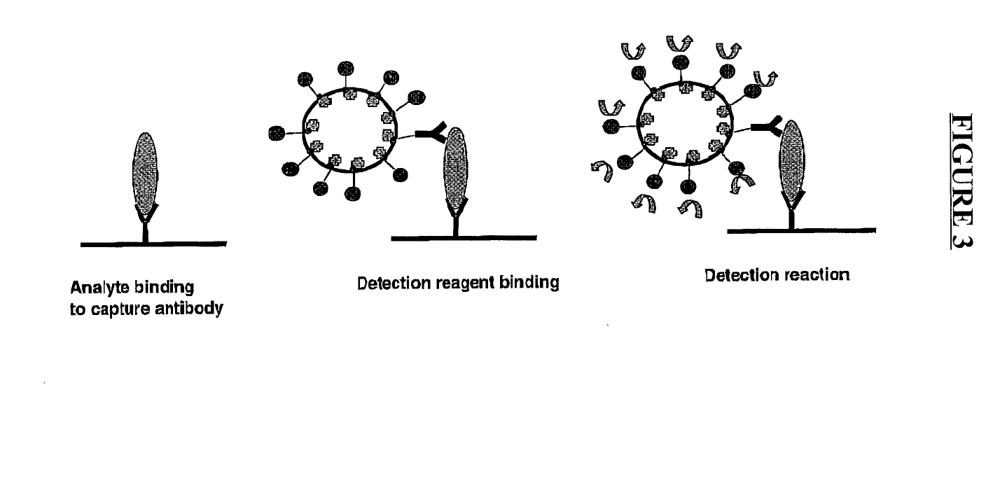

### DETAILED DESCRIPTION OF THE INVENTION

A branched bis-biotin/avidin/multiple label complex is provided wherein layers of bis-biotin and free biotin are attached to avidin or streptavidin and wherein two or more labeled moieties are attached to the outer layer of (strept)avidin via a biotin moiety. One or more specific binding pair members such as, for example, haptens, proteins, receptors and nucleic acids, is then bound to the complex, also to the outer layer of (strept)avidin via a biotin moiety.

The present branched bis-biotin/avidin/multiple label complex is distinguished from known compositions wherein multiple biotins are bound to a specific binding pair member by means of a bond or linking group. In such compositions, there is a distribution of compounds with varying number of biotins or each biotin molecule is bound to a separate functional group on the specific binding pair member. Accordingly, at least some part of the chain of atoms linking the biotins in the known compounds comprises the specific binding pair member.

Before proceeding further with a description of specific embodiments of the present invention, a number of terms will be defined and described in detail.

The term “avidin” is used herein generically and includes any biotinbinding protein other than an immunoglobulin that binds biotin, including both natural proteins and recombinant and genetically engineered proteins. The term includes the two common biotin-binding proteins known as “egg white or avian avidin” and “streptavidin.” Egg white or avian avidin, commonly referred to simply as avidin, is a protein that is a constituent of egg white and forms a non-covalent complex with biotin. Streptavidin is a protein isolated from the actino-bacterium Streptomyces avidinii and also forms a noncovalent complex with biotin. Other bacterial sources of biotin binding proteins are also known. Both egg white avidin and streptavidin are tetrameric proteins in which the biotin binding sites are arranged in pairs on opposite faces of the avidin molecule. Accordingly, both of the above avidins have the ability to bind to up to four molecules of biotin, either in the free form or in a derivative form and, thereby, form a “complex.” A derivative form of biotin results from the conjugation of biotin to another molecule. Because of the ability of avidin to bind a biotin derivative, avidin-biotin binding has been used in diagnostic assays, for example, to form reagent complexes either prior to or during an assay.

“Bis-biotin” refers to a compound comprising two biotinyl radicals connected together. Bis-biotin such as, for example, EZ-Link™ PEO-Biotin dimer, is available from Pierce of Rockford, Ill.. Also available from Pierce are as linking groups that can be used to connect the two biotinyl radicals The term “linking member” refers to a portion of a structure that connects two or more substructures.

As used herein, the term “conjugate” means a molecule comprised of two or more substructures bound together, generally through a linking member, to form a single structure. The binding is by means of an attaching group. For example, a specific binding pair member attached to a bis-biotin/avidin/multiple label complex is a bis-biotin/avidin/multiple label-specific binding pair member conjugate.

The term “member of a specific binding pair” (“sbp member”) refers to one of two different molecules having an area on the surface or in a cavity which specifically binds to and is, thereby, defined as complementary with a particular spatial and polar organization of the other molecule. A specific binding pair member may be any molecule that is able to specifically bind to another molecule. The members of the specific binding pair are often referred to as “ligand” and “receptor,” as later defined herein. The ligand and receptor will typically be members of an immunological pair such as antigen-antibody. Although other specific binding pairs such as hormones-hormone receptors, nucleic acid duplexes, IgG-protein A, polynucleotide pairs such as DNA-DNA, DNA-RNA and the like are not immunological pairs, they are included in the definition of sbp member for the purpose of describing this invention.

“Analyte” is the compound or composition to be detected. The analyte can be comprised of a member of a specific binding pair member. Representative analytes, by way of example and not limitation, include polypeptides and proteins, polysaccharides, nucleic acids and combinations thereof. Such combinations include components of bacteria, viruses, chromosomes, genes, mitochondria, nuclei, cell membranes and the like. The term “analyte” further includes oligonucleotide and polynucleotide analytes such as m-RNA, r-RNA, t-RNA, DNA, DNA-RNA duplexes, etc.

The analyte may be a molecule found directly in a sample such as biological tissue, including body fluids, from a host. The sample can be examined directly or may be pretreated to render the analyte more readily detectable by removing unwanted materials. The sample may be pretreated to separate or lyse cells; precipitate, hydrolyse or denature proteins; hydrolyze lipids; solubilize the analyte; or the like. Such pretreatment may include, without limitation, centrifugation; treatment of the sample with an organic solvent, for example, an alcohol, such as methanol; and treatment with detergents. The sample can be prepared in any convenient medium that does not interfere with an assay. An aqueous medium is preferred.

The analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay.

A “ligand” is any organic compound for which a receptor naturally exists or can be prepared.

The term “hapten” refers to a compound capable of binding specifically to corresponding antibodies, but does not itself act as an immunogen (or antigen) for preparation of the antibodies. Antibodies that recognize a hapten can be prepared against compounds comprised of the hapten linked to an immunogenic (or antigenic) carrier. Haptens are a subset of ligands.

“Ligand analog” means a modified ligand, an organic radical or analyte analog, usually of a molecular weight greater than 100, that can compete with the analogous ligand for a receptor, the modification providing means to join a ligand analog to another molecule. The ligand analog will usually differ from the ligand by more than replacement of a hydrogen with a bond which links the ligand analog to a hub or label, but need not. The ligand analog can bind to the receptor in a manner similar to the ligand. The analog could be, for example, an antibody directed against the idiotype of an antibody to the ligand.

As used herein, the term “receptor” (or “antiligand”) refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, protein A, complement component C1q and the like.

“Specific binding” refers to the specific recognition of one of two different molecules for the other compared to substantially less recognition of other molecules. Generally, the molecules have areas on their surfaces or in cavities, also referred to as “binding sites,” giving rise to specific recognition between the two molecules. Exemplary of specific binding are antibody-antigen interactions, enzyme-substrate interactions, polynucleotide interactions and so forth.

“Non-specific binding” refers to non-covalent binding between molecules that is relatively independent of specific surface structures. Non-specific binding may result from several factors including hydrophobic interactions between molecules.

The term “antibody” refers to an immunoglobulin that specifically binds to and is, thereby, defined as complementary with a particular spatial and polar organization of another molecule. The antibody can be monoclonal or polyclonal and can be prepared by techniques that are well-known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgGI, IgG2a, IgG2b and IgG3, IgM, etc. Fragments thereof may include Fab, Fv and F(ab′)₂, Fab′ and the like. In addition, aggregates, polymers and conjugates of immunoglobulins or their fragments can be used where appropriate, so long as binding affinity for a particular molecule is maintained.

The term “support” means a solid phase that is a porous or non-porous water insoluble material that can have any one of a number of shapes, such as strip, rod, plate, well, particle or bead. A wide variety of suitable supports are disclosed in U.S. Pat. Nos. 5,185,243; 4,868,104; and 4,959,303, which are incorporated herein by reference. The surface can be hydrophilic or capable of being rendered hydrophilic and includes inorganic powders such as silica, magnesium sulfate and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross-linked dextran, agarose, polyacrylate, polyethylene, polypropylene, poly(4-methylbutene), polystyrene, polymethacrylate, poly(ethylene terephthalate), nylon, poly(vinyl butyrate), etc., either used by themselves or in conjunction with other materials; and glass available as bioglass, ceramics, metals and the like. Natural or synthetic assemblies such as liposomes, phospholipid vesicles and cells can also be employed. Binding of sbp members to a support or surface, other than by use of the compounds of the present invention, may be accomplished by well-known techniques, commonly available in the literature. See, e.g., “Immobilized Enzymes,” Ichiro Chibata, Halsted Press, New York (1978) and Cuatrecasas, Biol. Chem. 245:3059 (1970).

“Signal amplification system” refers to one or more components, at least one component being a detectable label, that generates a detectable signal that relates to the amount of bound and/or unbound label, i.e., the amount of label bound or not bound to the compound being detected. The label is any molecule that produces or can be induced to produce a readable signal and may be, for example, a fluorescer, radio-label, enzyme, chemiluminescer or photosensitizer. Thus, the signal is detected and/or measured by detecting enzyme activity, luminescence, light absorbence or radioactivity as the case may be.

Suitable labels include, by way of illustration and not limitation, an enzyme for chemiluminescent detection or a fluorescent label for direct detection. Alternatively, the label may be a luminescent molecule, such as dioxetane, that may be activated by enzymes. In a preferred embodiment, the label is an enzyme, particularly, alkaline phosphatase and, more particularly, secreted alkaline phosphatase (“SEAP”), and the reporter substrate on which the enzyme acts is a dioxetane such as AMPPD®G. Other dioxetanes such as CSPD® and CDP-Star®, all available from Applied Biosystems in Bedford, Mass., may similarly be used. Dioxetane substrate is added and caused to decompose in the presence of the enzyme. On decomposition, the dioxetane reporter molecule releases light, which is read with a high resolution CCD camera, luminometer or scanning fluorescent reader.

The phrase “multiple label” means two or more labels.

An “assay” is a method for the determination of the presence or amount of an analyte.

The phrase “measuring the amount of an analyte” includes quantitative, semi-quantitative and qualitative methods, as well as all other methods for determining the amount of an analyte. For example, a method that merely detects the presence or absence of an analyte in a sample suspected of containing the analyte is considered to be included within the scope of the present invention. The terms “detecting” and “determining”, as well as other common synonyms for measuring, are contemplated within the scope of the present invention.

“Ancillary materials” refers to various ancillary materials that will frequently be employed in the assay in accordance with the present invention. For example, buffers will normally be present in the assay medium, as well as stabilizers for the assay medium and the assay components. Frequently, in addition to these additives, proteins such as albumins; organic solvents such as formamide; quaternary ammonium salts; polyanions such as dextran sulfate; surfactants, particularly non-ionic surfactants; binding enhancers, e.g., polyalkylene glycols; or the like may be included.

As mentioned above, one aspect of the present invention is a branched bis-biotin/avidin/multiple label complex. Another aspect is a branched bis-biotin/avidin/multiple label-specific binding pair member conjugate composition. When used as a reagent, the conjugate composition of the invention exhibits enhanced signal amplification because of the multiple labels. The functionality of the inventive branched bis-biotin/avidin/multiple label complex is further characterized in that its reaction with the specific binding pair member does not substantially affect the ability of the specific binding pair member to bind to its counterpart.

According to yet another aspect of the present invention, there is provided a method of polymerizing the branched bis-biotin/avidin/multiple label complex. The method comprises alternating additions of the biotin and avidin monomers to a support until the desired polymer is reached. The method, therefore, is a layer-bylayer building of the polymer. Because the method according to the present invention is a layer-by-layer building, the method is controllable and reproducible. In other words, polymerization always occurs and the results are consistent, i.e., identical polymer particles of certain reproducible size result each time the polymerization is repeated using the desired number of alternating additions. The method of the present invention differs from known prior art methods in that it comprises alternating additions of the monomers rather than a mixing of all monomers together at once. Additionally, the labels are bound to the final outer layer as the final step in polymerizing the complex of the present invention.

For example, a biotinylated double strand DNA or a layer of single biotin is placed on the solid support so as to link the polymer thereto during polymerization. Next, one of the monomers, for example, avidin or streptavidin, is added and incubated. Any unbound monomer is then washed out. Next a mixture of free biotin and bis-biotin is added. After allowing for incubation, any unbound biotin is washed out. These alternating additions of monomers are repeated the desired number of times, ending with a layer of avidin or streptavidin. Labels such as, for example, enzymes, are then attached to the outer (strept)avidin layer via a biotin moiety to form the branched bis-biotin/avidin/multiple label complex of the present invention. The result is a biotin/avidin/label complex having pure polymer layers and labels only on the surface, or outer layer. The advantage of having labels on the surface is that when using this complex in an assay where enzymes are used as the labels, the desired enzymatic reactions will occur because the enzymes, being located on the surface rather than within the polymer, are accessible. Moreover, the enzymes, if placed in each layer, may interfere with the polymerization itself.

Similar polymers may be made by this condensational-type polymerization where every monomer used is bi-functional. For example, nylon 6/6 may be polymerized from adipic acid and hexamethylene diamine using this method, as well as polyester from diacids (or activated diacids) and organic diols.

A specific binding pair member may be conjugated to the complex of the present invention, specifically to the outer (strept)avidin layer via a biotin moiety; therefore, another aspect of the present invention is a method of preparing a branched bis-biotin/avidin/multiple label-specific binding pair member conjugate composition for use in a specific binding assay. The method generally comprises the step of reacting the specific binding pair member with the branched bis-biotin/avidin/multiple label complex of the present invention. To free the resulting conjugate composition (also referred to as “polymer particles”) from the support, restriction digestion of the double stranded DNA is used or, if the polymer was attached using a single layer of biotin, then the polymer particles are photocleaved. Only the conjugate complex that can be cleaved from the support by restriction digestion or photocleavage will be cleaved. Any unspecifically bound complexes (i e., anything other than the polymer particles) remain on the support.

Accordingly, another aspect of the present invention concerns an improvement in an assay for an analyte that utilizes a reagent system comprising an avidin reagent and a biotin reagent. The improvement comprises using as the biotin reagent the bis-biotin/avidin/multiple label complex conjugated to a specific binding pair member. The conjugate composition of the present invention can be used in specific binding assays, utilizing a reagent comprised of a sbp member irreversibly attached to a label. The conjugate composition of the present invention provides for enhanced signal in the assay.

The conjugate composition of the present invention may be applied to most assays for the determination of an analyte that is a sbp member. In general, a sample suspected of containing an analyte is combined in an assay medium with a binding partner for the analyte. The binding of the binding partner to the analyte, if present, is detected. The assay can be performed either without separation (homogeneous) or with separation (heterogeneous) of any of the assay components or products. Heterogenous assays are preferred.

The present compositions may be used in homogeneous assays where the reactions can be carried out in solution phase. In these assays, any dissociation of a labeled sbp member into a free label can reduce the sensitivity of the assay because binding of an unlabeled sbp member can compete with binding of a labeled sbp member that, in turn, is related to the presence or amount of analyte to be determined. The stability achieved in the present invention provides numerous advantages for reagents used in assays. For example, a bis-biotin/avidin/multiple label complex in accordance with the present invention does not decompose into component parts, thus releasing, for example, a sbp member component.

Homogeneous immunoassays are exemplified by the EMIT™ assay products (Syva Company of San Jose, Calif.) disclosed in U.S. Pat. No. 3,817,837; immunofluorescence methods such as those disclosed in U.S. Pat. No. 3,996,345; enzyme channeling techniques such as those disclosed in U.S. Pat. No. 4,233,402; and other enzyme immunoassays such as the enzyme-linked immunosorbant assay (“ELISA”) disclosed in Yalow et al., J. Clin. Invest. 39:1157 (1960). The above disclosures are all incorporated herein by reference.

The present compositions find particular use in heterogeneous assays. Heterogeneous assays usually involve one or more separation steps and can be competitive or non-competitive. A variety of competitive and non-competitive heterogeneous assay formats are disclosed in U.S. Pat. No. 5,089,390, incorporated herein by reference. In a typical competitive heterogeneous assay, a support having an antibody for analyte bound thereto is contacted with a medium containing the sample and analyte analog conjugated to a detectable label such as an enzyme (the “conjugate”). Analyte in the sample competes with the conjugate for binding to the antibody. After separating the support and the medium, the label activity of the support or the medium is determined by conventional techniques and is related to the amount of analyte in the sample.

A typical non-competitive sandwich assay is an assay disclosed in U.S. Pat. No. 4,486,530, incorporated herein by reference. In this method, an immune sandwich complex is formed in an assay medium. The complex comprises the analyte, a first antibody (monoclonal or polyclonal) that binds to the analyte and a second antibody that binds to the analyte or a complex of the analyte and the first antibody. Subsequently, the immune sandwich complex is detected and is related to the amount of analyte in the sample. The immune sandwich complex is detected by virtue of the presence in the complex of a label wherein either or both the first antibody and the second antibody contain labels or substituents capable of combining with labels.

Sandwich assays find use for the most part in the detection of antigen and receptor analytes. In the assay, the analyte is bound by two antibodies specific for the analyte; therefore, the assay is also referred to as the “two-site immunometric assay.” In one approach, a first incubation of unlabeled antibody coupled to a support, otherwise known as the insolubilized antibody, is contacted with a medium containing a sample suspected of containing the analyte. After a wash and separation step, the support is contacted with a medium containing the second antibody, which generally contains a label, for a second incubation period. The support is again washed and separated from the medium and either the medium or the support is examined for the presence of label. The presence and amount of label is related to the presence or amount of the analyte. U.S. Pat. No. 4,474,878 and Re 29,169, the relevant disclosures of which are incorporated herein by reference, provide a more detailed discussion.

In a variation of the above sandwich assay, the sample in a suitable medium is contacted with labeled antibody for the analyte and incubated for a period of time. Then, the medium is contacted with a support to which is bound a second antibody for the analyte. After an incubation period, the support is separated from the medium and washed to remove unbound reagents. The support or the medium is examined for the presence of the label, which is related to the presence or amount of analyte. U.S. Pat. No. 4,098,876, the relevant disclosure of which is incorporated herein by reference, provides a more detailed discussion of this approach.

In another variation of the above, the sample, the first antibody bound to a support and the labeled antibody are combined in a medium and incubated in a single incubation step. Separation, wash steps and examination for label are as described above. U.S. Pat. No. 4,244,940, the relevant disclosure of which is incorporated herein by reference, provides a more detailed discussion of this approach.

The present invention has application to all of the above assays.

The present branched bis-biotin/avidin/multiple label complexes and branched bis-biotin/avidin/multiple label-specific binding pair conjugate compositions can be utilized in any of the known situations wherein a mono-biotin reagent is employed. For example, in U.S. Pat. No. 4,298,685, the relevant disclosure of which is incorporated herein by reference, there is disclosed an assay for an analyte that is an antigen, hapten or other biological substance. A sample suspected of containing the analyte is mixed with antibody for the analyte, which is bound to biotin, and with a known amount of the analyte labeled with an enzyme. After the competitive complexation of the antibody with the labeled analyte and the analyte in the sample, avidin immobilized on an inert support is added. The avidin binds to the biotin and causes the complex to be precipitated. After separation of the solid and liquid phases, enzyme activity of one or both is measured, the amount thereof being related to the amount of analyte in the sample. In accordance with the present invention, the branched bis-biotin/avidin/multiple label complex of the present invention can be substituted for the above biotin reagent where the specific binding pair member of the bis-biotin compound is the antibody for the analyte.

The present invention also finds use in agglutination assays employing plastic particles such as latex particles. In a typical agglutination assay of this type, a sbp member is bound to the surface of the plastic particles. This sbp member is capable of binding to an analyte. Usually, the sbp member is an antigen and the analyte is an antibody. The particles are incubated with a medium suspected of containing the analyte. The presence of the analyte causes the particles to agglutinate and the extent of agglutination is measured by known means and related to the presence or amount of the analyte. The present invention can be used to prepare the particles having the sbp member bound thereto. The particles can be incubated with a bis-biotin/avidin/multiple label complex of the present invention wherein the sbp member of the bis-biotin complex is the sbp member that binds to the analyte. The resulting particles have the sbp member bound thereto in an irreversible manner.

The present bis-biotin/avidin multiple label complexes and branched bis-biotin/avidin/multiple label-specific binding pair conjugate compositions can also be utilized in assays for polynucleotides such as DNA, RNA and so forth. In accordance with the present invention, a bis-biotin/avidin/multiple label complex as described herein may be used for forming the labeled probe. Other ways in which the instant bis-biotin/avidin/multiple label complexes can be used in polynucleotide assays will be suggested to those skilled in the art with reference to the present disclosure.

Another aspect of the present invention is a kit comprising a bis-biotin/avidin/multiple label complex and a specific binding pair member that is, or may be, conjugated to the complex. To enhance the versatility of the subject invention, the kit reagents can be provided in packaged combination, in the same or separate containers, in liquid or lyophilized form so that the ratio of the reagents provides for substantial optimization of the method and assay. The reagents may each be in separate containers or various reagents can be combined in one or more containers depending on the cross-reactivity and stability of the reagents.

The kit can further include other separately packaged reagents for conducting an assay such as additional sbp members, ancillary reagents such as additional signal producing system members, e.g., an ancillary enzyme substrate, and so forth. The relative amounts of the various reagents in the kits can be varied widely to provide for concentrations of the reagents that substantially optimize the reactions that need to occur and to further substantially optimize the sensitivity of the assay. Under appropriate circumstances, one or more of the reagents in the kit can be provided as a dry powder, usually lyophilized, including excipients that, upon dissolution, will provide for a reagent solution having the appropriate concentrations for performing a method or assay in accordance with the present invention. The kit can further include a written description of a method in accordance with the present invention as described above.

The invention is demonstrated further by the following illustrative examples.

### EXAMPLES

General Discussion

The presence of the target analyte in cells can be ascertained by usual immunological procedures applicable to such determinations. As discussed above, a number of useful procedures are known. Three such procedures that are especially useful utilize either the target analyte (“TA”) labeled with a detectable label, a DNA that binds a specific mRNA labeled with a detectable label, antibody Ab₁ labeled with a detectable label or antibody Ab₂ labeled with a detectable label. The three procedures may be summarized as follows:

TA*+Ab₁=TA*Ab₁ (1)

TA+Ab₁*=TA Ab₁* (2)

TA+Ab₁+Ab₂*=TA Ab₁ Ab₂* (3)

The procedures and their applications are all familiar to those skilled in the art and accordingly may be utilized within the scope of the present invention. The “competitive procedure” (1) is described in U.S. Pat. Nos. 3,654,090 and 3,850,752. The “sandwich procedure” (3) is described in U.S. Pat. No. 4,016,043 and Re 31,006.

In each instance, the target analyte forms complexes with one or more antibodies or binding partners, and one member of the complex is labeled with a detectable label. The fact that a complex is formed and the amount thereof can be determined by one of the above-described methods for detecting labels.

It will be seen from the above that a characteristic property of Ab₂ is that it will react with Ab₁. That is because Ab₁ raised in one mammalian species has been used in another species as an antigen to raise Ab₂.

Synthesis of a Branched DNA/RNA Hybrid-specific Antibody with Enhanced Signal Amplification Power

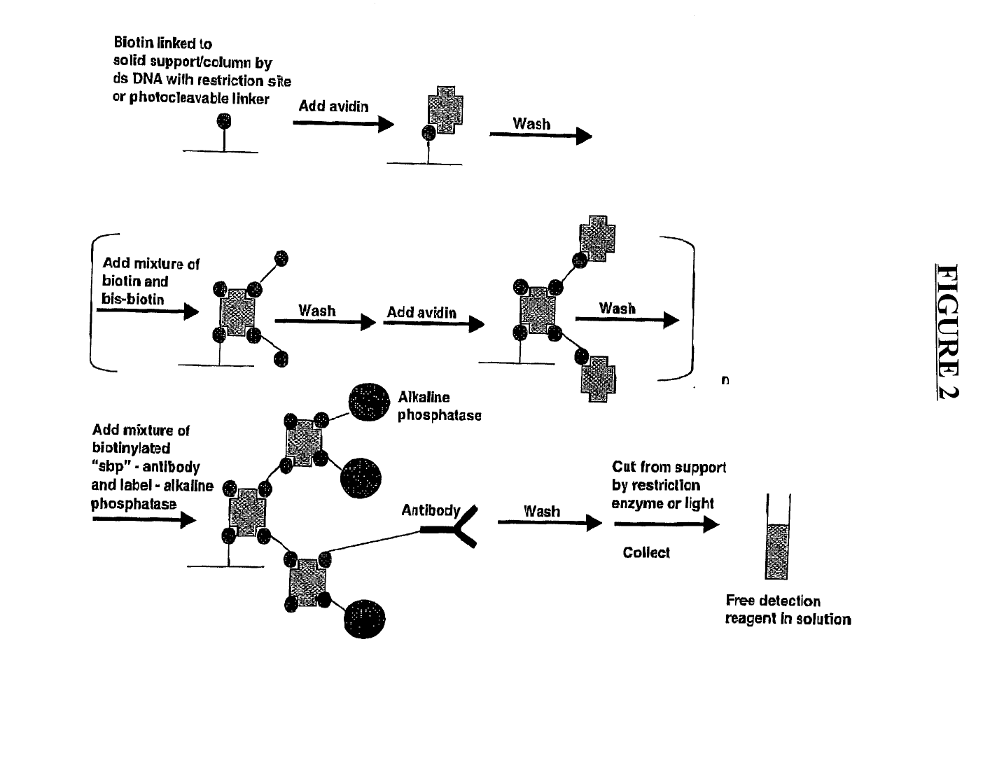

Referring to FIG. 2, a bis-biotin/avidin/multiple label-specific binding pair conjugate according to the present invention was prepared as follows:

(1) A biotinylated double stranded DNA linker with two restriction enzyme sites was attached to a support loaded with biotinylated linkers with the goal of keeping the distance between them enough to prevent substantial cross-linking of the forming polymers. In this example, the support was a column. Note that while a double stranded DNA linker was used here to hold the biotin to the surface, EZ-Link™ NHS-PC-LC-Biotin available from Peirce may also be used. Using EZ-Link™ instead of double stranded DNA allows for cleavage by light, i.e., it is a photocleavable connection.

(2) The column was loaded with streptavidin in excess to saturate all biotin. Each streptavidin molecule binds to four biotin molecules.

(3) The unbound streptavidin was washed out.

(4) The column was loaded with a mixture of free biotin and bis-biotin (from Pierce) and incubated.

(5) Unbound biotin was washed out.

(6) The column was loaded with streptavidin in excess to saturate all biotin and incubated.

(7) Unbound streptavidin was washed out.

(8) Steps (4)-(7) were repeated several times to reach a designated number of layers in the forming polymer particles.

(9) The column was loaded with biotinylated alkaline phosphatase and DNA/RNA hybrid-specific antibody in the desired proportion and incubated.

(10) Unbound protein was washed out.

(11) When sufficient enzyme/antibody/streptavidin complex had been built up, the column was loaded with restriction enzyme and incubated in order to free the formed conjugate composition. If EZ-Link™ NHS-PC-LC-Biotin was used, then the conjugate composition may be cleaved using light.

(12) Formed polymer particles were washed out.

(13) Non-specifically bound streptavidin, bis-biotin, alkaline phosphatase and antibody were retained in the column.

The result is polymer particles of the branched bis-biotin/(strept)avidin/multiple label-specific binding pair conjugate composition of the present invention. This composition is a highly specific reagent with high signal amplification results. The formed polymer particles may be used as the detection reagent in an mRNA detection assay. The polymer particles synthesized with corresponding antibody can also be used in any type of ELISA assay or DNA/RNA detection assay.

While the invention has been described and illustrated by reference to various specific material, procedures and examples, it is understood that the invention is not restricted to the particular material, combinations of material and procedures selected for that purpose. Numerous variations of such details can be implied as will be appreciated by those skilled in the art.

What is claimed is:

1. A method of preparing a branched bis-biotin/avidin/multiple label complex, comprising:
(a) alternating additions of biotin and avidin monomers, ending with an outer layer of avidin; and
(b) attaching labels to the outer layer of avidin.

2. The method of claim 1, wherein the bis-biotin is a mixture of free biotin and bis-biotin.

3. The method of claim 1, wherein the avidin is streptavidin.

4. The method of claim 1, wherein the labels are selected from the group consisting of enzymes, fluorescers, radio-labels, chemiluminescers and photosensitizers.

5. The method of claim 4, wherein the labels are enzymes.

6. The method of claim 1, wherein each of the labels is attached to the outer layer of avidin by a single biotin molecule.

7. A method of preparing a bis-biotin/avidin/multiple label complex conjugate composition, comprising:
(a) alternating additions of biotin and avidin monomers, ending with an outer layer of avidin;
(b) attaching labels to the outer layer of avidin; and
(c) attaching a specific binding pair member to the outer avidin layer of the complex.

8. The method of claim 7, wherein the bis-biotin is a mixture of free biotin and bis-biotin.

9. The method of claim 7, wherein the avidin is streptavidin.

10. The method of claim 7, wherein the labels are selected from the group consisting of enzymes, fluorescers, radio-labels, chemiluminescers and photo sensitizers.

11. The method of claim 10, wherein the labels are enzymes.

12. The method of claim 7, wherein each of the labels is attached to the outer layer of avidin by a single biotin molecule.

13. The method of claim 7, wherein the specific binding pair member is conjugated to the outer avidin layer by a single biotin molecule.

14. The method of claim 7, wherein the specific binding pair member is selected from the group consisting of haptens, antigens, receptors and polynucleotides.

15. A branched bis-biotin/avidin/multiple label complex, comprising layers of biotin and avidin monomers, wherein multiple labels are attached to an outer layer of avidin.

16. A branched bis-biotin/avidin/multiple label complex-specific binding pair member conjugate composition, comprising layers of biotin and avidin monomers, wherein multiple labels are attached to an outer layer of avidin and wherein one or more specific binding pair members is attached to the outer layer of avidin.

17. In an assay for an analyte involving a biotin-avidin reagent system, the improvement comprising using the branched bis-biotin/avidin/multiple label complex-specific binding pair member conjugate composition of claim 16 as the reagent system.

18. A kit comprising:
(a) the bis-biotin/avidin/multiple label complex of claim 15; and
(b) a specific binding pair member.
